(19) United States
(12) Patent Application Publication
(10) Pub. No.: US 2019/0005587 A1
(43) Pub. Date: Jan. 3, 2019

(54) DEVICE, SYSTEM, AND METHOD FOR OPTIMIZING A PATIENT FLOW

(71) Applicant: KONINKLIJKE PHILIPS N.V., EINDHOVEN (NL)

(72) Inventors: Reza SHARIFI SEDEH, Malden, MA (US); Yugang JIA, Winchester, MA (US); Daniel Robert ELGORT, New York, NY (US)

(21) Appl. No.: 16/066,348

(22) PCT Filed: Dec. 21, 2016
(86) PCT No.: PCT/IB2016/057855

§ 371 Date: Jun. 27, 2018

Related U.S. Application Data
(60) Provisional application No. 62/272,204, filed on Dec. 29, 2015.

(51) Int. Cl. G06Q 40/08; G16H 10/60

(52) U.S. Cl. CPC G06Q 40/08; G16H 10/60

(57) ABSTRACT

A device, system, and method optimizes a patient flow. The method is performed at a device of a healthcare organization, the healthcare organization having a healthcare network including a plurality of healthcare providers. The method includes determining a step in a patient flow for a patient of a primary care physician (PCP) associated with the healthcare network based upon first information relative to the patient. The method includes determining a referral of a healthcare provider to perform the step based upon the first information and second information relative to a region associated with the patient and the healthcare organization. The method includes determining whether the referral is acceptable based upon third information relative to the healthcare provider and the healthcare organization. The method includes generating a recommendation including the referral for the PCP when the referral is acceptable.

### BACKGROUND INFORMATION

A healthcare organization may be utilized by a patient to receive healthcare from available healthcare providers within a network of the healthcare organization. There are a variety of ways that the healthcare organization may be organized to provide services to the patient. One approach is through an accountable care organization (ACO). The ACO has a network of healthcare providers including primary care physicians (PCP), specialists, etc. The PCP may be in charge of the healthcare plan to be provided to a plurality of patients. Thus, patients may receive treatment from the PCP or from other healthcare providers from within the ACO network via a referral from the PCP.

The ACO may operate as a value-based approach where a bundled-payment or capitation is used in contrast to conventional healthcare organizations that operate in a volume-based approach where a fee for each service is used. Using the value-based approach, the ACO utilizes the healthcare providers who have associated with each other to provide coordinated quality care to the patients. That is, a patient utilizing the ACO may be charged a bundled cost for an overall treatment which may comprise of a plurality of services or treatments. In this manner, the PCP may manage a patient flow and coordinate the patient care within the ACO network.

For an ACO to determine the patient flow in a way that optimizes the quality of care and maximizes financial benefit, the ACO must manage the healthcare providers including hospitals of the healthcare network of the ACO. Specifically, the referrals to be used in the patient flow must be determined properly to minimize or eliminate certain actions such as unnecessary referrals, sub-optimal referrals, referrals out of the healthcare network of the ACO, etc. These actions may unnecessarily reduce the quality of care to the patient and increase the cost to the ACO that is responsible for all associated costs in the overall treatment which reduces the financial benefits to the ACO (since the patient or third party payer is only responsible for the bundled cost).

### SUMMARY

The exemplary embodiments are directed to a method performed at a device of a healthcare organization, the healthcare organization having a healthcare network including a plurality of healthcare providers. The method includes determining a step in a patient flow for a patient of a primary care physician (PCP) associated with the healthcare network based upon first information relative to the patient, determining a referral of a healthcare provider to perform the step based upon the first information and second information relative to a region associated with the patient and the healthcare organization, determining whether the referral is acceptable based upon third information relative to the healthcare provider and the healthcare organization and generating a recommendation including the referral for the PCP when the referral is acceptable.

The exemplary embodiments are also directed to a device of a healthcare organization, the healthcare organization having a healthcare network including a plurality of healthcare providers. The device having a transceiver communicating via a communications network, the transceiver configured to receive first information relative to the patient, second information relative to a region associated with the patient and the healthcare organization, and third information relative to the healthcare provider and the healthcare organization and a processor determining a step in a patient flow for a patient of a primary care physician (PCP) associated with the healthcare network based upon the first information, the processor determining a referral of a healthcare provider to perform the step based upon the first information and the second information, the processor determining whether the referral is acceptable based upon the third information, the processor generating a recommendation including the referral for the PCP when the referral is acceptable.

The exemplary embodiments are also directed to a non-transitory computer readable storage medium with an executable program stored thereon, wherein the program instructs a microprocessor to perform operations. The operations include determining a step in a patient flow for a patient of a primary care physician (PCP) associated with a healthcare network of a healthcare organization based upon first information relative to the patient, determining a referral of a healthcare provider to perform the step based upon the first information and second information relative to a region associated with the patient and the healthcare organization, determining whether the referral is acceptable based upon third information relative to the healthcare provider and the healthcare organization and generating a recommendation including the referral for the PCP when the referral is acceptable.

### BRIEF DESCRIPTION OF THE DRAWINGS

FIG. 1 shows a system according to the exemplary embodiments.

FIG. 2 shows a server of FIG. 1 according to the exemplary embodiments.

FIG. 3 shows a method for optimizing a patient flow according to the exemplary embodiments.

### DETAILED DESCRIPTION

The exemplary embodiments may be further understood with reference to the following description and the related appended drawings, wherein like elements are provided with the same reference numerals. The exemplary embodiments are related to a device, a system, and a method for optimizing a patient flow. The patient flow may relate to a sequence of services or treatments to be provided to a patient of a primary care physician (PCP) of a healthcare network of a healthcare organization where the healthcare organization may be an accountable care organization (ACO). The exemplary embodiments provide a mechanism in which the services or treatments utilizing a referral are determined for the PCP to optimize the patient flow with regard to a quality of care to the patient and financial benefit to the healthcare organization.

The exemplary embodiments relate to providing affordable healthcare to patients via a value-based approach. Specifically, to reduce healthcare costs and improve care, the ACO provides this value-based approach in which a bundled fee is charged to the patient (or a third-party payer) for a given overall treatment to be provided regardless of the steps and treatments that are involved in the course of the overall treatment. The ACO has a healthcare network comprising healthcare providers such as primary care physicians (PCP), specialists, other care providers (e.g., non-doctor provider such as a physical therapist), etc. or healthcare locations such as hospitals, labs, etc. who provide coordinated quality healthcare to the patient (e.g., Medicare patient). Accordingly, in treating a patient, the ACO offers a group of providers that agree on assuming a collective responsibility for delivering and coordinating healthcare for a patient.

In this process of providing healthcare to a patient with the ACO, the PCP plays an important role in managing a patient flow and coordinating a patient care within the healthcare network of the ACO. Specifically, the PCP may diagnose and/or determine the overall treatment as well as the referrals for treatments involved in the overall treatment to be performed. Therefore, with particular regard to referrals, the PCP must decide to which healthcare provider (e.g., a specialist) at a particular location (e.g., a hospital) to send a patient to receive a treatment as a step in a patient flow involved in the overall treatment.

The ACO is responsible for the overall treatment, associated costs, and outcomes of the patients while the patient (or a third party payer) is responsible for a bundled cost for the overall treatment. Accordingly, the ACO optimizes its financial performance through keeping the patient to only or mostly healthcare providers within the healthcare network of the ACO (without referring the patient to other ACOS, out-network hospitals, out-network specialists, etc.). The Centers for Medicare and Medicaid Services (CMS) hold the ACO responsible for all the associated costs in the overall treatment and outcome of the patient treated by healthcare providers inside and outside the healthcare network of the ACO. Accordingly, among various other factors such as organizing the healthcare network of the ACO to include an optimal set of PCPs, an optimal patient flow in performing the overall treatment results in an increased quality of care and maximized financial performance for the ACO. That is, to achieve financial and clinical goals, the ACO needs to understand patient flows and optimize care delivery for the patient flow (overall and each step) within the resource constraints of the healthcare network of the ACO. Specifically, the ACO must understand which hospital or specialist that a PCP should recommend for a patient given clinical conditions and resource constraints of the healthcare network of the ACO. Therefore, the ACO may better coordinate healthcare within the healthcare network and, consequently, improve quality of care for the patients and reduce financial costs of the ACO.

The exemplary embodiments are configured to provide a mechanism to optimize the patient flow for patients of PCPs associated with the healthcare network of the ACO. Specifically, the mechanism of the exemplary embodiments may be provided to the PCPs of the healthcare network of the ACO. The mechanism may include an automated process of determining a step to be performed in the patient flow such as an ensuing step and also determining a referral in completing this ensuing step. The mechanism of the exemplary embodiments may generate a recommendation to the PCP for these determined results (e.g., which hospital or which specialist). Using various types of referral data including claims information, clinical information, and utilization information, the exemplary embodiments may properly perform the determination. More specifically, the determination may be made to optimize objectives of the patient and the ACO such as utilizing healthcare providers who are associated with the healthcare network of the ACO (hereinafter referred to as “in network”) while minimizing use of healthcare providers who are not associated with the healthcare network of the ACO (hereinafter referred to as “out network”). Thus, the exemplary embodiments may provide a manner in which the PCP may optimize the patient flows within the ACO.

It should be noted that the description herein relates to a component of the ACO that performs a plurality of functionalities in determining a referral for a patient within a patient flow. However, this is only exemplary. The exemplary embodiments may also be implemented in various other devices. For example, a device of the PCP may perform the functionalities when information is available in executing the functionalities. It should also be noted that the description herein relates to optimizing the operation of the ACO. However, this too is only exemplary. The exemplary embodiments may also be utilized in optimizing any healthcare organization, particularly through optimizing a patient flow for patients of PCPs in the healthcare network of the healthcare organization.

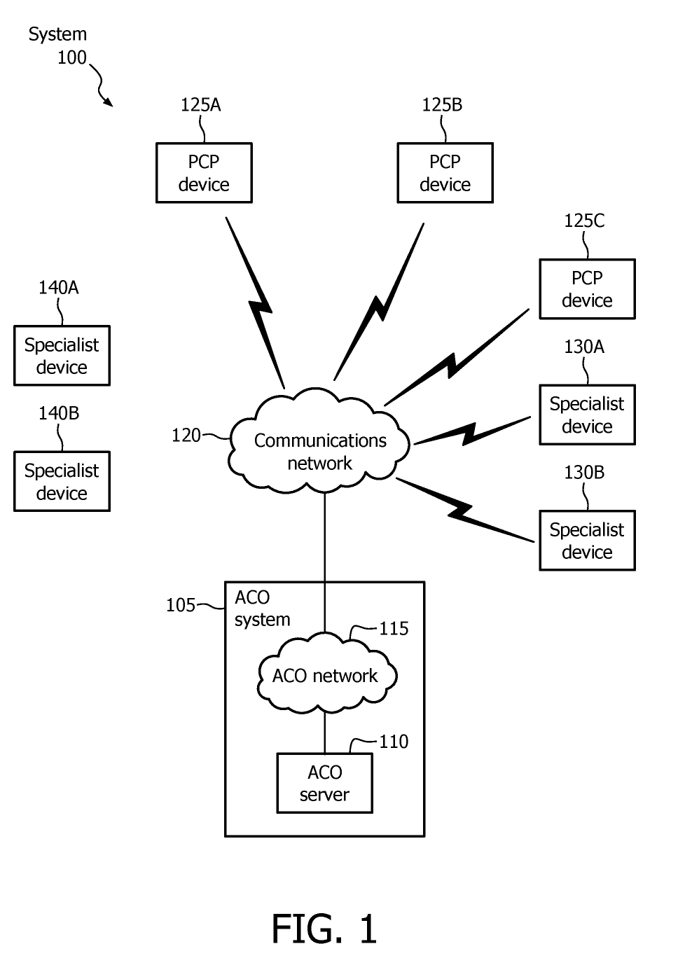

FIG. 1 shows a system 100 according to the exemplary embodiments. The system 100 relates to a plurality of PCPs who may be associated (e.g., in network PCPs and in network specialists) or unassociated (e.g., out network specialists) with an ACO healthcare organization. Specifically, the system 100 for example includes an ACO system 105 in which in network PCPs and in network specialists may utilize the healthcare network associated with the ACO system 105. As will be described in further detail below, the system 100 may include a plurality of in network PCPs using PCP devices 125A-C, a plurality of in network specialists using specialist devices 130A-B, and a plurality of out network specialists using specialist devices 140A-B.

The system 100 further includes a communications network 120 that is communicatively connected to an ACO network 115 of the ACO system 105. Accordingly, the PCP devices 125A-C and the specialist devices 130A-B utilized by healthcare providers of the healthcare network of the ACO may be authorized to access the ACO system 105 and any data repositories such as a list and description of the healthcare providers included in the healthcare network (e.g., for referral purposes). The communications network 120 may represent any single or plurality of networks used by the PCP devices 125A-C and the specialist devices 130A-B to communicate with the ACO system 105. For example, if the PCP devices 125A-C are computers used at an office, the communications network 120 may include an office network in which the PCP devices 125A-C may initially connect. The office network may connect to a network of an Internet service provider to connect to the Internet. Subsequently, through the Internet, a connection may be established with the ACO network 115. It should be noted that the communications network 120 and all networks that may be included therein may be any type of network. For example, the communications network 120 may be a local area network (LAN), a wide area network (WAN), a virtual LAN (VLAN), a WiFi network, a HotSpot, a cellular network (e.g., 3G, 4G, Long Term Evolution (LTE), etc.), a cloud network, a wired form of these networks, a wireless form of these networks, a combined wired/wireless form of these networks, etc. The communications network 120 may also represent one or more networks that are configured to connect to one another to enable the data to be exchanged among the components of the system 100.

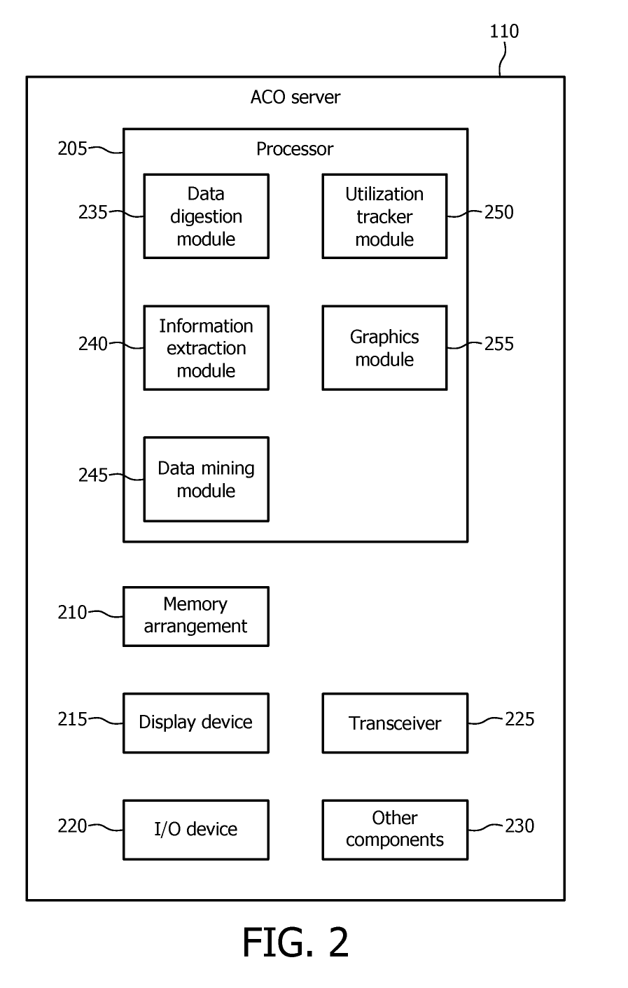

The ACO system 105 includes the ACO network 115 and an ACO server 110. The ACO network 115 of the ACO system 105 may enable the PCP devices 125A-C and the specialist devices 130A-B to access available information provided by the ACO system 105 such as the healthcare network and healthcare providers of the ACO. The ACO network 115 may be configured with an authentication or authorization feature (e.g., an authentication, authorization, and accounting (AAA) procedure (via a AAA server)), that requires identification information to be provided that is used as the basis for granting or denying the access. The ACO network 115 may be a proprietary network using protocols such as the various types described above in a wireless or wired manner. It should be noted that the ACO network 115 may include a variety of components (not shown) to enable these functionalities. For example, the ACO network 115 may include the ACO server 110, data repositories, a router, a switch center, a network management arrangement, etc. The ACO server 110 will be described in further detail below with regard to FIG. 2.

As noted above, the PCP devices 125A-C and the specialist devices 130A-B may be computing devices utilized by healthcare providers who are associated with the healthcare network of the ACO such as in network PCPs. The PCP devices 125A-C and the specialist devices 130A-B may represent any electronic device that is configured to perform the functionalities corresponding to use associated with a healthcare provider. For example, the PCP devices 125A-C and the specialist devices 130A-B may be a portable device such as a tablet, a laptop, etc. or a client stationary device such as a desktop terminal. The PCP devices 125A-C and the specialist devices 130A-B may include the necessary hardware to perform the various procedures and/or treatments as well as the necessary software associated with the procedures/treatments and patient information. The PCP devices 125A-C and the specialist devices 130A-B may also include the required connectivity hardware, software, and firmware (e.g., transceiver) to establish a connection with the communications network 120 to further establish a connection with the ACO network 115.

The system 100 may also represent a localized area. That is, the system 100 may show the PCP devices 125A-C and the specialist devices 130A-B who have agreed upon providing the value-based healthcare treatment to patients of the ACO who are within a defined geographic area. The geographic area may be defined using a variety of factors. For example, the geographic area may be determined for a particular patient and an acceptable distance from the home of the patient. Thus, the PCP devices 125A-C and the specialist devices 130A-B may be determined based upon a specific patient. In another example, the geographic area maybe determined based upon areas designated by an administrator or manager of the ACO. Thus, the PCP devices 125A-C and the specialist devices 130A-B may be selected regardless of the patients. Therefore, the PCP devices 125A-C and the specialist devices 130A-B may be a first group of a plurality of groups of the ACO who have been designated the geographic area. It should be noted that the PCP devices 125A-C and the specialist devices 130A-B may be associated with one or more groups for patients of the ACO for respective geographic areas. It should also be noted that the number of PCP devices 125A-C and the specialist devices 130A-B illustrated in the system 100 of FIG. 1 is only exemplary. Those skilled in the art will understand that there may be any number of PCP devices and specialist devices. In fact, increased PCP devices and specialist devices may ensure that patients of the ACO who are within the defined geographic area may always be treated by healthcare providers who are associated with the healthcare network of the ACO.

The specialist devices 140A-B may be computing devices utilized by healthcare providers who are not associated with the healthcare network of the ACO such as out network specialists. The specialist devices 140A-B may be substantially similar to the specialist devices 130A-B. Thus, the specialist devices 140A-B may include the necessary hardware, software, and firmware. The specialist devices 140A-B are illustrated in the system 100 of FIG. 1 as not connected to the communications network 120. However, the specialist devices 140A-B may be configured for such a functionality such as connecting to the Internet. As noted above, the specialist devices 140A-B may be utilized by healthcare providers who are not associated with the ACO. Thus, the specialist devices 140A-B may be capable of connecting to the communications network 120 but incapable of connecting to the ACO network 115. However, it should be noted that the ACO network 115 may provide guest access to out network healthcare providers so that the in network healthcare providers may be identified and referred if necessary.

Also substantially similar to the PCP devices 125A-C and the specialist devices 130A-B, the specialist devices 140A-B may be healthcare providers who may be included within the defined geographic area. Thus, the specialist devices 140A-B may be within a geographically bound area to be considered for a referral if a need should arise. For example, the in network specialists for the defined geographic area may lack a particular specialty that is covered by a specialist utilizing one of the specialist devices 140A-B and not in the healthcare network of the ACO. It should again be noted that the number of the specialist devices 140A-B illustrated in the system 100 of FIG. 1 is only exemplary. Those skilled in the art will understand that there may be any number of specialist devices who are out network.

As described above, the ACO server 110 may be a component of the ACO system 105. FIG. 2 shows the ACO server 110 of FIG. 1 according to the exemplary embodiments. The ACO server 110 may provide a recommendation functionality for referrals to the in network PCPs of the healthcare network of the ACO system 105. Although the ACO server 110 is described as a network component (specifically a server), the ACO server 110 may be embodied in a variety of ways such as a portable device (e.g., a tablet, a smartphone, a laptop, etc.) or a client stationary device (e.g., a desktop terminal). The ACO server 110 may include a processor 205, a memory arrangement 210, a display device 215, an input and output (I/O) device 220, a transceiver 225, and other components 230 (e.g., an imager, an audio I/O device, a battery, a data acquisition device, ports to electrically connect the ACO server 110 to other electronic devices, etc.).

The processor 205 may be configured to execute a plurality of applications of the ACO server 110. As will be described in further detail below, the processor 205 may utilize a plurality of modules including a data digestion module 235, an information extraction module 240, a data mining module 245, a utilization tracker module 250, and a graphics module 250. The data digestion module 235 may ingest the information used by the other modules such as claims information, clinical information, and utilization information. The information extraction module 240 may evaluate the clinical information of the patient to determine an ensuing step of the patient flow of the overall treatment. The data mining module 245 may evaluate the claims information and the clinical information to determine a recommendation for a referral for the patient. The utilization tracker module 250 may determine whether to adopt the decision of the data mining module 240 such as through using the utilization information. The graphics module 250 may generate a graphical user interface for the recommendation.

It should be noted that the above noted applications and modules each being an application (e.g., a program) executed by the processor 205 is only exemplary. The functionality associated with the applications may also be represented as components of one or more multifunctional programs, a separate incorporated component of the ACO server 110 or may be a modular component coupled to the ACO server 110, e.g., an integrated circuit with or without firmware.

The memory 210 may be a hardware component configured to store data related to operations performed by the ACO server 110. Specifically, the memory 210 may store data related to the ingested information and the healthcare providers who are in network and out network. The display device 215 may be a hardware component configured to show data to a user while the I/O device 220 may be a hardware component that enables the user to enter inputs. For example, an administrator or manager of the ACO system 105 may maintain and update the functionalities of the ACO server 110 through user interfaces shown on the display device 215 with inputs entered with the I/O device 220. It should be noted that the display device 215 and the I/O device 220 may be separate components or integrated together such as a touchscreen. The transceiver 225 may be a hardware component configured to transmit and/or receive data such as the ingested information. That is, the transceiver 225 may enable the communication with other electronic devices directly or indirectly through the ACO network 115 and/or the communications network 120.

As noted above, the exemplary embodiments may provide a mechanism to determine a referral in a step of a patient flow related to a patient of an in network PCP. Specifically, the referral may initially relate to determining the step such as the type of treatment as well as the associated referral destination such as a hospital or specialist. Additionally, the exemplary embodiments may provide a mechanism to determine a feasibility of the referral with regard to the healthcare network of the ACO. Therefore, in performing these functionalities, the ACO server 110 may utilize the above noted modules.

The data digestion module 235 may ingest the referral data such as the claims information, the clinical information, and the utilization information. Specifically, using the transceiver 225, the data digestion module 235 may receive these different forms of information. For example, the claims information may relate to claims of the ACO and the claims of the regional CMS (corresponding to the defined geographic area of the system 100). Accordingly, the ACO claims may be received from data repositories of the ACO system 105 that may be connected to the ACO network 115 whereas the regional CMS claims may be received from data repositories of the CMS (not shown) that may be connected to the communications network 120. The data digestion module 235 may receive the claims information for analysis and formatting to be used by the ACO server 110, particularly the regional CMS claims information which may not be organized in a manner consistent with the ACO server 110. The claims information may be used to address a financial objective such as reducing a financial cost for the referral. In another example, the clinical information may relate to information already established for the patient and the patient flow in performing the overall treatment as well as any steps already performed. Accordingly, the clinical information may be received from the PCP device associated with the in network PCP of the patient. The clinical information may be used to address a quality of care objective such as improvement to the quality of care. In a further example, the utilization information may relate to information pending at the referral destinations of the healthcare providers in the healthcare network of the ACO (e.g., a load for a specialist at a particular hospital to not overload the hospital department or the specialist). That is, a schedule of the healthcare providers may be used in determining availability. Accordingly, the utilization information may be received from the healthcare providers of the healthcare network of the ACO. The utilization information may be used to address ACO constraints such as resource constraints.

It should be noted that the data digestion module 235 may ingest the information at a variety of times. For example, the data digestion module 235 may continuously or at predetermined time intervals update the claims and/or utilization information such that the claims and/or utilization information remains up-to-date. In another example, the data digestion module 235 may update the clinical information each time a recommendation is to be determined such that the more current clinical information of the patient flow may be used as a basis. It should also be noted that the ACO claims information may be readily available as this information may be stored in a data repository of the ACO system 105. However, the other information including the regional CMS claims information, the clinical information, and the utilization information may require a request, an authorization, a fee, etc.

The information extraction module 240 may evaluate the clinical information of the patient. More specifically, the information extraction module 240 may determine the ensuing step for the overall treatment in the current patient flow of the patient. In performing this evaluation, the information extraction module 240 may consider the overall treatment, the steps that have been performed, the remaining steps, a prior medical history of the patient, etc. The information extraction module 240 may therefore generate a result corresponding to an ensuing step.

The data mining module 245 may utilize data mining algorithms (e.g., machine learning methods, pattern recognition methods, process mining methods, etc.) on the claims information and the clinical information to determine an optimal selection for a referral destination (e.g., a hospital, a specialist, etc.). For example, the data mining module 245 may determine the pool of specialists who are capable of performing the treatment associated with the determined ensuing step for the patient flow. Specifically, the data mining module 245 may utilize the claims information and the clinical information through the data mining algorithms to perform these determinations as the capabilities may be extracted from this information. In another example, the data mining module 245 may determine which of the available specialists are in network as well as associated rates and fees to perform the treatment associated with the ensuing step in the patient flow. Specifically, the data mining module 245 may utilize the claims information through the data mining algorithms to perform this determination as this information may be extracted from the claims information. In a further example, the data mining module 245 may determine whether the in network specialists have the capability or capacity to perform the ensuing step of the patient flow. When such a capability is lacking, the data mining module 245 may ultimately determine that an out network specialist may be required but may select one based upon the rate/fee as indicated in the regional CMS claims information. Accordingly, the data mining module 245 may determine the referral for the ensuing step that improves a quality of care for the patient while minimizing financial costs to the ACO.

It is noted that the data mining module 245 may determine the result of the referral based upon an exclusive analysis. That is, the result of the referral may be formulated on the patient flow alone without consideration of outside factors. Therefore, according to the exemplary embodiments, the ACO server 110 may utilize the results of the data mining module 245 at face value or upon further analysis.

The utilization tracker module 250 may determine whether to adopt the result of the data mining module 245 for the ACO. The utilization tracker module 250 may communicate iteratively with the data mining module 245 to evaluate the output thereof. The utilization tracker module 250 may therefore provide the further analysis to the result of the data mining module 245. For example, the utilization tracker module 250 may consider the utilization database to properly distribute referrals to healthcare providers within the healthcare network of the ACO. Using this further analysis, the utilization tracker module 250 may accept or reject the result of the data mining module 245. The utilization tracker module 250 may also request the data mining module 245 to provide a further referral recommendation to be considered if a first recommendation is rejected. The utilization tracker module 250 may further request the information extraction module 240 to provide a further ensuing step recommendation if recommendation results from the data mining module 245 are unacceptable (e.g., to a degree such as after five, consecutive recommendations from the data mining module 245 are rejected).

In performing the further analysis, the utilization tracker module 250 may calculate financial risks, clinical risk, etc. For example, the utilization tracker module 250 may utilize processes to determine the optimal ensuing step for the patient flow and a corresponding referral recommendation that considers further factors such as an effect to the healthcare network and the ACO.

The graphics module 255 may generate a graphical user interface of the referral recommendation. The graphics module 255 may print the referral including the recommended ensuing step and the recommended referral destination for consideration by the PCP in detailing a course of action for the patient in performing the overall treatment.

Using the above modules, the ACO server 110 may be configured to perform the functionalities in determining a referral recommendation related to a patient flow for a patient of a PCP in the healthcare network of the ACO. For example, the specialist devices 130A-B and 140A-B in the system 100 of FIG. 1 may represent any destination for a referral such as an office of the specialist or a hospital where the specialist may be employed or work. Although only five referral destinations are shown where three are in network in the system 100 of FIG. 1, it should again be noted that there may be any number of referral destinations where there may also be any number of in network referral destinations and any number of out network referral destinations. Through analysis of the referral destinations, the overall treatment may relate to, for example, a hip replacement and an ensuing step in the patient flow for the hip replacement may be the surgery itself (via the information extraction module 240).

In a first example, the ACO server 110 may determine that the specialist utilizing the specialist device 130B is the optimal choice (via the data mining module 245) for this ensuing step in terms of quality of care (e.g., highest rated physician who performs this step) and healthcare cost (e.g., in network specialist). In selecting the specialist utilizing the specialist device 130B, the ACO server 110 may generate a score. The score may include a quality of care portion and a cost portion. Given that the objectives are satisfied, the score may reflect this aspect. An availability (via the utilization tracker module 250) may also be determined for the specialist utilizing the specialist device 130B and if available, may further increase the score. With a highest score from among the referral destinations, the specialist utilizing the specialist device 130B may be selected as the recommendation for the referral.

In a second example, the ACO server 110 may determine that the specialist utilizing the specialist device 140A is the optimal choice for the ensuing step in terms of quality of care. Accordingly, the specialist utilizing the specialist device 140A may have a highest quality of care portion for the score. The ACO server 110 may also determine that the specialist utilizing the specialist device 130A may rank slightly below the specialist 140A in terms of quality of care (e.g., within a predetermined tolerable threshold range). Accordingly, the specialist utilizing the specialist device 130A may have a quality of care portion for the score that is lower than the quality of care portion of the specialist utilizing the specialist device 140A. However, the ACO server 110 may further determine that the specialist utilizing the specialist device 130A being in network provides a significantly higher cost portion of the score than the specialist utilizing the specialist device 140A. The combined portions to generate the score may ultimately result in the specialist utilizing the specialist device 130A having a score greater than the specialist utilizing the specialist device 140A. An availability may again be determined for the specialist utilizing the specialist device 130A and, if available, may further increase the score. With a highest score (although a lower quality of care portion), the specialist utilizing the specialist device 130A may be selected as the recommendation for the referral.

In a third example, the above conditions in the second example in terms of relativity of the quality of care and the cost may be used. However, a severity of the difference between the specialists utilizing the specialist devices 130A and 140A may create a different result. For example, the specialist utilizing the specialist device 140A may have a quality of care portion that is significantly higher than the quality of care portion of the specialist utilizing the specialist device 130A. This significant difference in the quality of care portion may be still greater than any difference between the cost portion for the specialists utilizing the specialist devices 130A and 140A despite the specialist utilizing the specialist device 130A being in network while the specialist utilizing the specialist device 140A is out network. The combined portions to generate the score may ultimately result in the specialist utilizing the specialist device 140A having a score greater than the specialist utilizing the specialist device 130A. In this manner, the specialist utilizing the specialist device 140A may be selected as the recommendation for the referral.

It is noted that the utilization information may or may not include information related to out network referral destinations. The in network healthcare providers may constantly provide information to the ACO system 105 such that a schedule, a load, etc. of the healthcare provider may be updated in the utilization information. The out network healthcare providers may be incapable of providing this information to the ACO system 105. Therefore, the utilization tracker module 250 may not be configured to be used when selecting an out network referral destination. However, it is noted that the related data for the utilization information of the out network healthcare providers may be requested or determined (e.g., publicly available) for the functionality of the utilization tracker module 250 to be used even for out network healthcare providers.

In a fourth example, the above conditions in the second example may be used. However, upon utilizing the further analysis provided by the utilization tracker module 250, the availability of the specialist utilizing the specialist device 130A may be determined to be unavailable for at least a duration of time beyond an acceptable threshold. Accordingly, the utilization tracker module 250 may reject the referral recommendation of the data mining module 245 and request a further recommendation. As the other specialist utilizing the specialist device 140A is determined to be a next best recommendation, the data mining module 245 may provide this specialist as the further recommendation. However, the utilization tracker module 250 may determine that the associated cost in referring to the out network specialist utilizing the specialist device 140A is beyond an acceptable cost threshold. Accordingly, the utilization tracker module 250 may also reject this further referral recommendation. The data mining module 245 may accordingly determine a still further recommendation for the referral.

Accordingly, the exemplary embodiments provide the recommendation to the in network PCP of the patient who may always select the best choice of hospitals and specialists for a referral through considering the needs of the patient and the healthcare organization.

It should again be noted that the above description of the mechanism provided by the exemplary embodiments relating to operations performed by the ACO server 110 is only exemplary. Those skilled in the art will understand that the exemplary embodiments may also be embodied in various other components configured to perform the operations described herein for the ACO server 110. Thus, in a first exemplary embodiment, the operations of the modules 235-255 may be performed by a network component of the ACO system 105 such as the ACO server 110. That is, the operations may be performed remotely relative to the PCP device requesting the recommendation. As the operations are performed by the ACO server 110, the results may be used to update the data repositories storing the referral data. In a second exemplary embodiment, the operations of the modules 235-255 may be performed by an application executed on the PCP device. That is, the operations may be performed locally on the PCP device. Therefore, the PCP device may only require receiving the referral data from the ACO system 105. As the operations are performed by the PCP device, the results may subsequently be transmitted to update the data repositories storing the referral data.

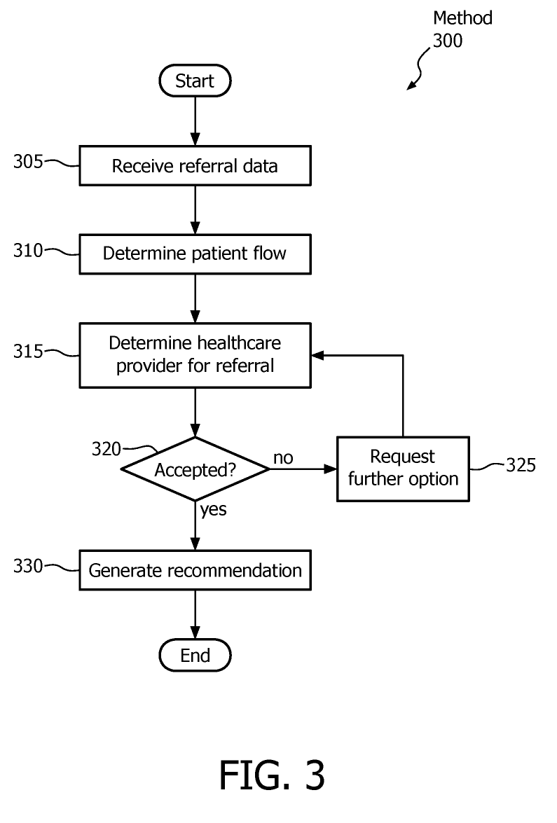

FIG. 3 shows a method 300 for optimizing a patient flow according to the exemplary embodiments. Specifically, the method 300 may relate to the mechanism of the exemplary embodiments in which a referral to a healthcare provider to perform a treatment for a patient of a PCP is determined. Accordingly, the method 300 may relate to the operations performed by the ACO server 110. The method 300 will be described with regard to the system 100 of FIG. 1 and the ACO server 110 of FIG. 2.

In step 305, the ACO server 110 receives the referral data. As described above, the referral data may include the claims information, the clinical information, and the utilization information. The clinical information may be used in determining an ensuing step of a patient flow for the patient. The claims information and the clinical information may be used in determining a recommended referral destination for the ensuing step. The utilization information may be used in providing a further analysis associated with the recommended referral destination. Thus, in step 310, the ACO server 110 via the information extraction module 240 determines the recommended ensuing step of the patient flow. In step 315, the ACO server 110 via the data mining module 245 determines the recommended healthcare provider to be referred in performing the ensuing step.

In step 320, the ACO server via the utilization tracker module 250 determines whether the recommended healthcare provider to perform the ensuing step is accepted or rejected. As discussed above, the utilization information may provide data used as part of a further analysis in determining the feasibility of the recommended healthcare provider to be used as a referral. If the recommendation generated by the data mining module 245 is rejected, the ACO server 110 continues the method 300 to step 325 where a further recommendation is requested. The ACO server 110 returns the method 300 to step 315. When the ACO server 110 has accepted the recommendation generated by the data mining module 245, the ACO server 110 continues the method 300 to step 330. In step 330, the recommendation is generated and provided to the PCP of the patient.

The exemplary embodiments provide a device, system, and method of generating a recommendation for a referral to have a step of a patient flow performed for a patient of a PCP in a healthcare network of a healthcare organization. The exemplary embodiments may utilize various sources of data to determine the step, determine the healthcare provider for the referral, and evaluate further considerations in utilizing the referral which may affect the patient in terms of a quality of care and the healthcare organization in terms of a cost.

Those skilled in the art will understand that the above-described exemplary embodiments may be implemented in any suitable software or hardware configuration or combination thereof. An exemplary hardware platform for implementing the exemplary embodiments may include, for example, an Intel x86 based platform with compatible operating system, a Windows platform, a Mac platform and MAC OS, a mobile device having an operating system such as iOS, Android, etc. In a further example, the exemplary embodiments of the above described method may be embodied as a computer program product containing lines of code stored on a computer readable storage medium that may be executed on a processor or microprocessor. The storage medium may be, for example, a local or remote data repository compatible or formatted for use with the above noted operating systems using any storage operation.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

1. A method, comprising:
receiving, by a server of an accountable care organization (ACO), a request for a recommendation from a user device of a healthcare professional;
at the ACO server, the ACO having a healthcare network including a plurality of healthcare providers:
determining a step in a patient flow for a patient of a primary care physician (PCP) associated with the healthcare network based upon first information relative to the patient;
determining a referral of a healthcare provider to perform the step based upon the first information and second information relative to a region associated with the patient and the healthcare organization;
determining whether the referral is acceptable based upon third information relative to the healthcare provider and the healthcare organization;
generating a recommendation including the referral for the PCP when the referral is acceptable; and
sending the recommendation to the user device of the healthcare professional.

2. The method of claim 1, wherein the first information is clinical information, wherein the second information is claims information, and wherein the third information is utilization information.

3. The method of claim 2, wherein the claims information includes first claims information relative to the healthcare organization and second claims information relative to regional Centers for Medicare and Medicaid Services (CMS).

4. The method of claim 2, wherein the utilization information includes appointment information of the healthcare provider.

5. The method of claim 1, further comprising:
at the ACO server:
determining a further referral of a further healthcare provider to perform the step based upon the first and second information.

6. The method of claim 5, further comprising:
at the ACO server:
determining a first score for the referral;
determining a second score for the further referral;
comparing the first and second scores; and
determining the first score is greater than the second score,
wherein the first and second scores are based upon a quality of care component relative to the patient and a cost component relative to the healthcare organization.

7. The method of claim 5, wherein the further referral is determined when the referral is unacceptable.

8. (canceled)

9. The method of claim 1, wherein the referral is one of a specialist and a hospital.

10. (canceled)

11. A server of an accountable care organization (ACO), the ACO having a healthcare network including a plurality of healthcare providers, comprising:
a transceiver communicating via a communications network, the transceiver configured to receive first information relative to the patient, second information relative to a region associated with the patient and the healthcare organization, and third information relative to the healthcare provider and the healthcare organization; and
a processor receiving a request for a recommendation from a user device of a healthcare professional, the processor determining a step in a patient flow for a patient of a primary care physician (PCP) associated with the healthcare network based upon the first information, the processor determining a referral of a healthcare provider to perform the step based upon the first information and the second information, the processor determining whether the referral is acceptable based upon the third information, the processor generating a recommendation including the referral for the PCP when the referral is acceptable, the processor sending the recommendation to the user device of the healthcare professional.

12. The server of claim 11, wherein the first information is clinical information, wherein the second information is claims information, and wherein the third information is utilization information.

13. The server of claim 12, wherein the claims information includes first claims information relative to the healthcare organization and second claims information relative to regional Centers for Medicare and Medicaid Services (CMS).

14. The server of claim 12, wherein the utilization information includes appointment information of the healthcare provider.

15. The server of claim 11, wherein the processor further determines a further referral of a further healthcare provider to perform the step based upon the first and second information.

16. The server of claim 15, wherein the processor further determines a first score for the referral, determines a second score for the further referral, compares the first and second scores, and determines the first score is greater than the second score, wherein the first and second scores are based upon a quality of care component relative to the patient and a cost component relative to the healthcare organization.

17. The server of claim 15, wherein the further referral is determined when the referral is unacceptable.

18. (canceled)

19. The server of claim 11, wherein the referral is one of a specialist and a hospital.

20. A non-transitory computer readable storage medium with an executable program stored thereon, wherein the program instructs a microprocessor to perform operations, comprising:
determining a step in a patient flow for a patient of a primary care physician (PCP) associated with a healthcare network of a healthcare organization based upon first information relative to the patient;
determining a referral of a healthcare provider to perform the step based upon the first information and second information relative to a region associated with the patient and the healthcare organization;
determining whether the referral is acceptable based upon third information relative to the healthcare provider and the healthcare organization; and
generating a recommendation including the referral for the PCP when the referral is acceptable.
